(19) United States
(12) Patent Application Publication
(10) Pub. No.: US 2024/0374234 A1
(43) Pub. Date: Nov. 14, 2024

(54) AUTOMATIC CLINICAL WORKFLOW THAT RECOGNIZES AND ANALYZES 2D AND DOPPLER MODALITY ECHOCARDIOGRAM IMAGES FOR AUTOMATED CARDIAC MEASUREMENTS AND GRADING OF MITRAL VALVE AND TRICUSPID VALVE REGURGITATION SEVERITY

(71) Applicants: EKO.AI PTE. LTD, Singapore (SG); MedStar Health, Inc., Columbia, MD (US)

(72) Inventors: James Otis HARE, II, Singapore (SG); Su Ping Carolyn LAM, Singapore (SG); Yoran HUMMEL, Singapore (SG); Zhubo JIANG, Singapore (SG); Matthew FROST, Singapore (SG); Federico Miguel ASCH, Singapore (SG)

(21) Appl. No.: 18/315,390

(22) Filed: May 10, 2023

(51) Int. Cl. A61B 8/08

(52) U.S. Cl. CPC A61B 8/0883; A61B 8/488; A61B 8/5223

(57) ABSTRACT

An automated workflow performed by software executing on at least one processor includes receiving a plurality of echocardiogram images of a heart. The plurality of echocardiogram (echo) images are separated according to 2D images and Doppler modality images. The 2D images are classified by view type, including PLAX, A2C, and A4C. The Doppler modality images are classified by region, including CW (Continuous Wave). Regions of interest in the 2D images are segmented images to produce segmented 2D images, including PLAX, A2C, and A4C segmented images. The Doppler modality images are segmented to generate waveform traces to produce segmented Doppler modality images. Both the segmented 2D images and the segmented Doppler modality images are used to calculate measurements of cardiac features of the heart. A grade of MR or TR severity is generated by comparing the calculated measurements to cardiac guidelines. At least one report is output showing the calculated measurements.

### TECHNICAL FIELD

The disclosed embodiments relate to image classification for disease prediction, and more specifically, to an automatic clinical workflow that recognizes and analyzes 2D and Doppler modality echocardiogram images for automated measurements and grading of Mitral Valve and Tricuspid Valve regurgitation severity.

### BACKGROUND

Cardiovascular disease including heart failure is a major health problem accounting for about 30% of human deaths worldwide. Heart failure is also the leading cause of hospitalization in adults over the age of 65 years. Echocardiography is an important diagnostic aid in cardiology for the morphological and functional assessment of the heart. In a typical patient echocardiogram (echo) examination, a clinician called a sonographer places an ultrasound device against the patient's chest to capture a number of 2D images/videos of the patients' heart. Reflected sound waves reveal the inner structure of the heart walls and the velocities of blood flows. The ultrasound device position is varied during an echo exam to capture different anatomical sections as 2D slices of the heart from different viewpoints or views. The clinician has the option of adding to these 2D images a waveform captured from various possible modalities including continuous wave Doppler, m-mode, pulsed wave Doppler and pulsed wave tissue Doppler. The 2D images/videos and Doppler modality images are typically saved in DICOM (Digital Imaging and Communications in Medicine) formatted files. Although the type of modality is partially indicated in the metadata of the DICOM file, the ultrasound device position in both the modality and 2D views, which is the final determinant of which cardiac structure has been imaged, is not.

After the patient examination, a clinician/technician goes through the DICOM files, manually annotates heart chambers and structures like the left ventricle (LV) and takes measurements of those structures. The process is reliant on the clinicians' training to recognize the view in each image and make the appropriate measurements. In a follow up examination, a cardiologist reviews the DICOM images and measurements, compares them to memorized guideline values and makes a diagnosis based on the interpretation made from the echocardiogram.

The current workflow process for analyzing DICOM images, measuring cardiac structures in the images and determining, predicting and prognosticating heart disease is highly manual, time-consuming and error-prone. Because the workflow process is so labor intensive, more than 95% of the images available in a typical patient echocardiographic study are never annotated or quantified. The view angle or Doppler modality type by which an image was captured is typically not labelled, which means the overwhelming majority of stored DICOMs from past patient studies and clinical trials do not possess the basic structure and necessary identification of labels to allow for machine learning on this data.

There has been a recent proposal for automated cardiac image interpretation to enable low-cost assessment of cardiac function by non-experts. Although the proposed automated system holds the promise of improved performance compared to the manual process, the system has several shortfalls. One shortfall is that the system only recognizes 2D images. In addition, although the proposed system may distinguish between a normal heart and a diseased heart, the proposed system is incapable of distinguishing hearts having similar-looking diseases. Consequently, the number of heart diseases identified by the proposed system is very limited and requires manual intervention to identify other types of heart diseases.

For example, heart failure has been traditionally viewed as a failure of contractile function and left ventricular ejection fraction (LVEF) has been widely used to define systolic function, assess prognosis and select patients for therapeutic interventions. However, it is recognized that heart failure can occur in the presence of normal or near-normal EF: so-called “heart failure with preserved ejection fraction (HFPEF)” which accounts for a substantial proportion of clinical cases of heart failure. Heart failure with severe dilation and/or markedly reduced EF: so-called “heart failure with reduced ejection fraction (HFREF)” is the best understood type of heart failure in terms of pathophysiology and treatment. The symptoms of heart failure may develop suddenly ‘acute heart failure’ leading to hospital admission, but they can also develop gradually. In addition, transthoracic echocardiography is a common imaging modality to screen for mitral valve regurgitation (MR) and tricuspid valve regurgitation (TR). Multi-parametric evaluation of MR and TR severity is recommended by international reference guidelines. Quantitative parameters are preferred, as they provide clear cutoff points for grading. However, application of multiple parameters is time consuming and prone to error and result in MR and TR severity interpretation variability. Timely diagnosis, categorization of heart failure subtype-HFREF or HFPEF, grading of MR and TR regurgitation severity, and improved risk stratification are critical for the management and treatment of heart failure, but the proposed system does not address this.

The proposed system is also incapable of generating a prognosis based on the identified heart disease and would instead require a cardiologist to manually form the prognosis. The proposed system is further incapable of structuring the automated measurements and labelled views across multiple sources of data, to enable training and validation of disease prediction algorithms across multiple remote patient cohorts.

Accordingly, there is a need for an improved and fully automatic clinical workflow that recognizes and analyzes both 2D and Doppler modality echocardiographic images for automated measurements and grading of mitral valve regurgitation (MR) and tricuspid valve regurgitation (TR) severity.

### BRIEF SUMMARY

The disclosed embodiments provide methods and systems for an automated workflow performed by a software component executing at least one processor. Aspects of disclosed embodiments include receiving a plurality of echocardiogram images of a heart. The plurality of echocardiogram (echo) images are separated according to 2D images and Doppler modality images. The 2D images are classified by view type, including PLAX, A2C, and A4C. The Doppler modality images are classified by region, including continuous wave focused on mitral regurgitation (CWMR) or continuous wave on tricuspid regurgitation (CWTR). Regions of interest in the 2D images are segmented images to produce segmented 2D images, including PLAX, A2C, and A4C segmented images. The Doppler modality images are segmented to generate waveform traces to produce segmented Doppler modality images. Both the segmented 2D images and the segmented Doppler modality images are used to calculate measurements of cardiac features of the heart. A grade of MR or TR severity is generated by comparing the calculated measurements to cardiac guidelines. At least one report is output showing the calculated measurements.

According to the method and system disclosed herein, the disclosed embodiments use machine learning to recognize and analyze both 2D and Doppler modality echocardiographic images for automated measurements and diagnosis of MR or TR severity, and the system can be deployed in workstation or mobile-based ultrasound point-of-care systems.

### BRIEF DESCRIPTION OF SEVERAL VIEWS OF THE DRAWINGS

FIGS. 1A-1C are diagrams illustrating embodiments of a system for implementing an automated clinical workflow that recognizes and analyzes both 2D and Doppler modality Echocardiographic images for automated measurements and the diagnosis, prediction and prognosis of heart disease.

FIG. 2 illustrates architectural layers of the echo workflow engine.

FIG. 3A is a flow diagram illustrating one embodiment of a process for performed by the echo workflow engine to automatically recognize and analyze both 2D and Doppler modality echo images to images to perform automated measurements and measurements and grading of mitral valve regurgitation (MR) and tricuspid valve regurgitation (TR) severity.

FIGS. 3B, 3C and 3D are diagrams illustrating MR jet area ratio (JAR) detection, MR vena contracta (VC) detection, and continuous wave doppler density (CWDD) detection for CWMR or CWTR, respectively.

FIG. 3F is a diagram illustrating an example report of mitral valve regurgitation severity.

FIG. 3E is a diagram illustrating an example report of tricuspid valve regurgitation severity.

FIG. 4A is a flow diagram illustrating details of the process for automatically recognizing and analyze both 2D and Doppler modality echo images to perform automated measurements and the diagnosis, prediction and prognosis of heart disease according to one embodiment.

FIG. 4B is a flow diagram illustrating advanced functions of the echo workflow engine.

FIG. 5A is a diagram illustrating an example 2D echo image.

FIG. 5B is a diagram illustrating an example Doppler modality image.

FIGS. 6A-6K are diagrams illustrating some example view types automatically classified by the echo workflow engine.

FIG. 7 is a diagram illustrating an example 2D image segmented to produce annotations indicating cardiac chambers, and an example Doppler modality image segmented to produce a mask and trace waveform.

FIGS. 8A and 8B are diagrams illustrating examples of finding systolic/diastolic end points.

FIGS. 9A and 9B are diagrams illustrating processing of an imaging window in a 2D echo image and the automated detection of out of sector annotations.

FIGS. 10A and 10B are diagrams graphically illustrating structural measurements automatically generated from annotations of cardiac chambers in a 2D image, and velocity measurements automatically generated from annotations of waveforms in a Doppler modality.

FIG. 11 is a diagram graphically illustrating measurements of global longitudinal strain that were automatically generated from the annotations of cardiac chambers in 2D images.

FIG. 12A is a diagram graphically illustrating an example set of best measurement data based on largest volume cardiac chambers and the saving of the best measurement data to a repository.

FIG. 12B is a diagram graphically illustrating the input of automatically derived measurements from a patient with normal LV EF measurements into a set of rules to determine a conclusion that the patient has normal diastolic function, diastolic dysfunction, or indeterminate.

FIG. 13 is a diagram graphically illustrating the output of classification, annotation and measurement data to an example JSON file.

FIG. 14 is a diagram illustrating a portion of an example report showing highlighting values that are outside the range of international guidelines.

FIG. 15 is a diagram illustrating a portion of an example report of Main Findings that may be printed and/or displayed by the user.

FIG. 16A is a diagram showing a graph A plotting PCWP and HFpEF scores.

FIG. 16B is a diagram showing a graph B plotting CV mortality or hospitalization for HF.

### DETAILED DESCRIPTION

The disclosed embodiments relate to an automatic clinical workflow that recognizes and analyzes 2D and Doppler modality Echocardiographic images for automated measurements and grading of mitral valve regurgitation (MR) and tricuspid valve regurgitation (TR) severity. The following description is presented to enable one of ordinary skill in the art to make and use the invention and is provided in the context of a patent application and its requirements. Various modifications to the disclosed embodiments and the generic principles and features described herein will be readily apparent. The disclosed embodiments are mainly described in terms of particular methods and systems provided in particular implementations. However, the methods and systems will operate effectively in other implementations. Phrases such as “disclosed embodiment”, “one embodiment” and “another embodiment” may refer to the same or different embodiments. The embodiments will be described with respect to systems and/or devices having certain components. However, the systems and/or devices may include more or less components than those shown, and variations in the arrangement and type of the components may be made without departing from the scope of the invention. The disclosed embodiments will also be described in the context of particular methods having certain steps. However, the method and system operate effectively for other methods having different and/or additional steps and steps in different orders that are not inconsistent with the disclosed embodiments. Thus, the present invention is not intended to be limited to the embodiments shown, but is to be accorded the widest scope consistent with the principles and features described herein.

The disclosed embodiments provide method and system for implementing a software-based automatic clinical workflow using machine learning that recognizes and analyzes both 2D and Doppler modality Echocardiographic images for automated measurements and grading of mitral valve regurgitation (MR) and tricuspid valve regurgitation (TR) severity, and which can be deployed in workstation or mobile-based ultrasound point-of-care systems.

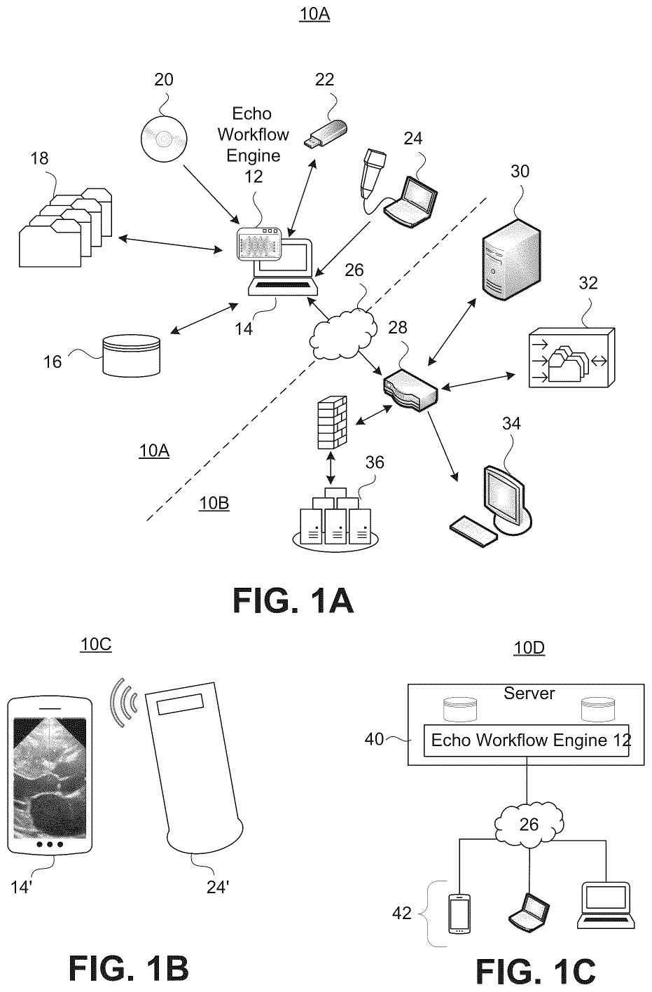

FIGS. 1A-1C are diagrams illustrating embodiments of a system for implementing an automated clinical workflow that recognizes and analyzes both 2D and Doppler modality Echocardiographic images for automated measurements and the diagnosis, prediction and prognosis of heart disease. FIG. 1A shows a basic standalone configuration for the automated clinical workflow system 10A and a connected configuration 10B. The automated clinical workflow 10A is primarily implemented as a software application, referred to as echo workflow engine 12, that executes on a computer 14 operating in a standalone setting, disconnected from other devices on network 26. The computer 14 may be implemented in any form factor including a workstation, desktop, notebook, laptop server or tablet capable of running an operating system, such as Microsoft Windows® (e.g., Windows 7®, Windows 10®), Apple macOS®, Linux®, Apple iOS®, Android®, and the like.

The computer 14 may include typical hardware components (not shown) including a processor, input devices (e.g., keyboard, pointing device, microphone for voice commands, buttons, touchscreen, etc.), output devices (e.g., a display device, speakers, and the like), and wired or wireless network communication interfaces (not shown) for communication. The computer 14 may include internal computer-readable media, such as memory (not shown) containing computer instructions comprising the echo workflow engine 12, which implements the functionality disclosed herein when executed by one or more computer processors.

The computer 14 may further include local internal storage for storing one or more databases 16 and an image file archive 18. In one embodiment, the contents of the image file archive 18 include echocardiogram image files (also referred to herein as echo images), which in some embodiments may be stored in DICOM (Digital Imaging and Communications in Medicine) format.

In one embodiment, the computer 14 is in communication with peripheral devices such a point-of-care (POC) device 25, an ultrasound imaging device 24, or both. The POC device 25 is capable of measuring cardiac biomarkers in POC environments such as an emergency room, intensive care unit, physician's office, an ambulance, a patient setting, and remote emergency sites.

In one embodiment, the computer 14 is in communication with peripheral devices such an ultrasound imaging device 24 that captures echocardiogram images of a patient's organ (e.g., a heart), which may then be stored as a patient study using the database 16 and image file archive 18. For example, the computer 14 may be located in a hospital or clinical lab environment where Echocardiography is performed as a diagnostic aid in cardiology for the morphological and functional assessment of the heart. During a typical patient echocardiogram exam (referred to as a study), a sonographer or technician places the ultrasound imaging device 24 against the patient's chest to capture 2D echo images/videos of the heart to help diagnose the particular heart ailment. Measurements of the structure and blood flows are typically made using 2D slices of the heart and the position of the ultrasound imaging device 24 is varied during an echo exam to capture different anatomical sections of the heart from different viewpoints. The technician has the option of adding to these 2D echo images a waveform captured from various possible modalities including: continuous wave Doppler, m-mode, pulsed wave Doppler and pulsed wave tissue Doppler. The 2D images and Doppler waveform images may be saved as DICOM files. Although the type of modality is sometimes indicated in the metadata of the DICOM file, the 2D view is not.

The computer 14 may further include removable storage devices such as an optical disk 20 and/or a flash memory 22 and the like for storage of the echo images. In some embodiments, the removable storage devices may be used as an import source of echo images and related data structures into the internal image file archive 18, rather than or in addition to, the ultrasound imaging device 24. The removable storage devices may also be used as an archive for echocardiogram data stored in the database 16 and/or the image file archive 18.

FIG. 1A also shows an advanced optional embodiment. referred to as connected configuration 10B, where the computer 14 may be connected through the network 26 and a router 28 to other DICOM based devices, such as DICOM servers 30, network file share devices 32, echo workstations 34, and/or cloud storage services 36 hosting DICOM files. In the connected configuration 10B, several possible interactions with the database 16 and the image file archive 18 are possible, as described below.

One possible interaction is to use the cloud storage services 36 as an internal archive. In case of very large archives consisting of large amounts of DICOM files, the computer 14 may not have sufficient storage to host all files and the echo workflow engine 12 may be configured to use external network storage of the cloud storage services 36 for file storage.

Another possible interaction is to use the cloud storage services 36 as an import source by i) selecting a DICOM file set or patient study, which includes the DICOM and Doppler waveforms images and patient data and examination information. The patient study may also be selected by a reserved DICOMDIR file instance, from which the patient, exams and image files are read.

Yet a further possible interaction is to use the DICOM servers 30, the network file share devices 32, echo workstations 34, and/or DICOM clients (of FIG. 1C) acting as DICOM servers (workstations with modalities CFind, CMove and CStore) in order to retrieve patients, exams and images by performing a CFind operation, followed by a CMove operation, to request the remote device to send the images resulting from the CFind operation.

Referring now to FIG. 1B, a handheld configuration 10C for the automated clinical workflow system is shown. In this embodiment, the computer 14 of FIG. 1A is implemented as a handheld device 14′, such as a tablet or a mobile phone, connected to a wired or wireless portable ultrasound scanner probe 24′ that transmits echo images to the handheld device 14′. In such embodiments, the echo workflow engine 12 may be implemented as an application executed by the handheld device 14′.

FIG. 1C illustrates a software as a service (Saas) configuration 10D for the automated clinical workflow. In this embodiment, the echo workflow engine 12 is run on a server 40 that is in communication over the network 26 with a plurality of client devices 42. In this embodiment, the server 40 and the echo workflow engine 12 may be part of a third-party service that provides automated measurements and the diagnosis, prediction and prognosis of heart disease to client devices (e.g., hospital, clinic, or doctor computers) over the Internet. It should be understood that although the server 40 is shown as a single computer, it should be understood that the functions of server 40 may be distributed over more than one server. In an alternative embodiment, the server 40 and the echo workflow engine 12 of FIG. 1C may be implemented as a virtual entity whose functions are distributed over multiple client devices 42. Likewise, it should be understood that although the echo workflow engine 12 is shown as a single component in each embodiment, the functionality of the echo workflow engine 12 may be separated into a greater number of modules/components.

Conventionally, after a patient examination where echo images are captured stored, a clinician/technician goes through the DICOM files, manually annotates heart chambers and structures and takes measurements, which are presented in a report. In a follow up examination, a doctor will review the DICOM images and measurements, compare them to memorized guideline values and make a diagnosis. Such a process is reliant on the clinicians' training to recognize the view and make the appropriate measurements so that a proper diagnosis can be made. Such a process is error-prone and time consuming.

According to the disclosed embodiments, the echo workflow engine 12 mimics the standard clinical practice, processing DICOM files of a patient by using a combination of machine learning, image processing, and DICOM workflow techniques to derive clinical measurements, diagnose specific diseases, and prognosticate patient outcomes, as described below. While an automated solution to echo image interpretation using machine learning has been previously proposed, the solution only analyzes 2D echo images and not Doppler modality waveform images. The solution also mentions disease prediction, but only attempts to handle two diseases (hypertrophic cardiomyopathy and cardiac amyloidosis) and the control only compares normal patients to diseased patients.

The echo workflow engine 12 of the disclosed embodiments, however, improves on the automated solution by utilizing machine learning to automatically recognize and analyze not only 2D echo images but also Doppler modality waveform images. The echo workflow engine 12 is also capable of comparing patients having similar-looking heart diseases (rather than comparing normal patients to diseased patients), and automatically identifies additional diseases, including both heart failure with reduced ejection fraction (HFrEF) and heart failure with preserved ejection fraction (HFpEF). HFrEF is known as heart failure due to left ventricular systolic dysfunction or systolic heart failure and occurs when the ejection fraction is less than 40%. HFpEF is a form of congestive heart failure where the amount of blood pumped from the heart's left ventricle with each beat (ejection fraction) is greater than 50%. Finally, unlike the proposed automated solution, the echo workflow engine 12 automatically generates a report with the results of the analysis for medical decision support, including the grading of mitral valve regurgitation (MR) and tricuspid valve regurgitation (TR) severity.

Multi-parametric evaluation of MR and TR is a comprehensive assessment of the severity and prognosis of mitral valve and tricuspid valve regurgitation. Mitral valve regurgitation (MR) and tricuspid valve regurgitation (TR) are heart valve diseases that occur when the mitral or tricuspid valve does not close properly, allowing blood to flow backward through the valve, which can put extra strain on the heart. This evaluation involves the assessment of several parameters, including the severity of aortic stenosis, left ventricular function, valvular morphology, and the presence of associated comorbidities.

There are several conventional methods to assess the severity of MR and TR using echocardiography. One way is to measure the area of the regurgitant jet, which is the stream of blood that flows backward through the valve. The area of the jet can be used to estimate the amount of blood that is leaking back through the valve. Vena contracta (VC) is the narrowest portion of the regurgitant jet in mitral regurgitation (MR) or tricuspid regurgitation (TR). VC is formed by the convergence of the regurgitant jet as it flows through the regurgitant orifice. The VC can be measured using echocardiography and used to assess the severity of MR or TR. The VC is a reliable marker of MR or TR severity because it is directly proportional to the regurgitant orifice area (ROA). The ROA is the area of the opening in the mitral valve that allows blood to flow backward from the ventricle to the atrium. A larger ROA indicates more severe MR or TR. VC can also be used to assess the severity of MR or TR in patients with eccentric or non-symmetrical jets. The VC can be used to measure the width of the jet at its narrowest point, which can help to determine the severity of MR or TR. If MR or TR is severe, it can cause the heart to become enlarged and weak. Currently, the severity of MR and TR is graded on a scale of 1 to 4, with 1 being the mildest and 4 being the most severe.

Traditional echocardiography to evaluate MR and TR is highly manual, time consuming, error-prone, limited to specialists, and involves long waiting times. However, the Artificial Intelligence (AI) approached described herein allows fully automated, fast and reproducible echocardiographic image analysis; turning a manual process of over 30 minutes, and over 250 user clicks, into an AI-automated process taking 2 minutes, 1 click, with 0% variability. Such AI-enabled echocardiographic interpretation therefore not only increases efficiency and accuracy, but also opens the door to decision support for non-specialists.

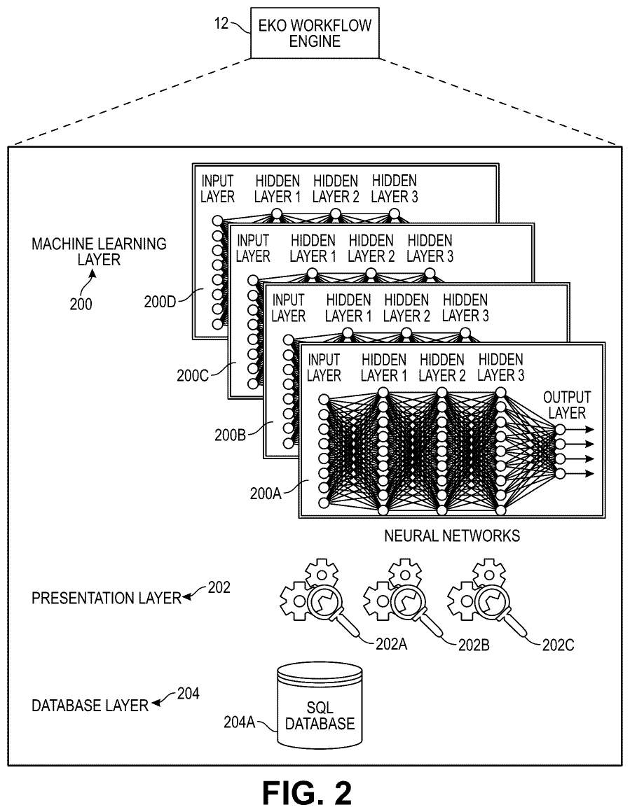

FIG. 2 illustrates architectural layers of the echo workflow engine 12. In one embodiment, the echo workflow engine 12 may include a number of software components such as software servers or services that are packaged together in one software application. For example, the echo workflow engine 12 may include a machine learning layer 200, a presentation layer 202, and a database layer 204.

The machine learning layer 200 comprises several neural networks to process incoming echo images and corresponding metadata. The neural networks used in the machine learning layer may comprise a mixture of different classes or model types. In one embodiment, machine learning layer 200 utilizes a first set of one or more neural networks 200A to classify 2D images by view type, and uses a second set of neural networks 200B to both extract features from Doppler modality images and to use the extracted features to classify the Doppler modality images by region (the neural networks used to extract features may be different than the neural network used to classify the images). The first set of neural networks 200A and the second set of neural networks 200B may be implemented using convolutional neural network (CNN) and may be referred to as classification neural networks or CNNs.

Additionally, a third set of neural networks 200C, including adversarial networks, are employed for each classified 2D view type in order to segment the cardiac chambers in the 2D images and produce segmented 2D images. A fourth set of neural networks 200D are used for each classified Doppler modality region in order to segment the Doppler modality images to generate waveform traces. In additional embodiments, the machine learning layer 200 may further include a set of one or more prediction CNNs for disease prediction and optionally a set of one or more prognosis CNNs for disease prognosis (not shown). The third and fourth sets of neural networks 200C and 200D may be implemented using CNNs and may be referred to as segmentation neural networks or CNNs.

In machine learning, a CNN is a class of deep, feed-forward artificial neural network typically used for analyzing visual imagery. Each CNN comprises an input and an output layer, as well as multiple hidden layers. In neural networks, each node or neuron receives an input from some number of locations in the previous layer. Each neuron computes an output value by applying some function to the input values coming from the previous layer. The function that is applied to the input values is specified by a vector of weights and a bias (typically real numbers). Learning in a neural network progresses by making incremental adjustments to the biases and weights. The vector of weights and the bias are called a filter and represents some feature of the input (e.g., a particular shape).

The machine learning layer 200 operates in a training mode to train each of the CNNs 200A-200D prior to the echo workflow engine 12 being placed in an analysis mode to automatically recognize and analyze echo images in patient studies. In one embodiment, the CNNs 200A-200D may be trained to recognize and segment the various echo image views using thousands of echo images from an online public or private echocardiogram DICOM database. In one embodiment, the CNNs are trained for both automated view identification and automated annotation of relevant MR and TR views in the echo images.

The presentation layer 202 is used to format and present information to a user. In one embodiment, the presentation layer is written in HTML 5, Angular 4 and/or JavaScript. The presentation layer 202 may include a Windows Presentation Foundation (WPF) graphical subsystem 202A for implementing a lightweight browser-based user interface that displays reports and allows a user (e.g., doctor/technician) to edit the reports. The presentation layer 202 may also include an image viewer 202B (e.g., a DICOM viewer) for viewing echo images, and a python server 202C for running the CNN algorithms and generating a file of the results in JavaScript Object Notation (JSON) format, for example.

The database layer 204 in one embodiment comprises a SQL database 204A and other external services that the system may use. The SQL database 204A stores patient study information for individual patient studies input to the system. In some embodiments, the database layer 204 may also include the image file archive 18 of FIG. 1A.

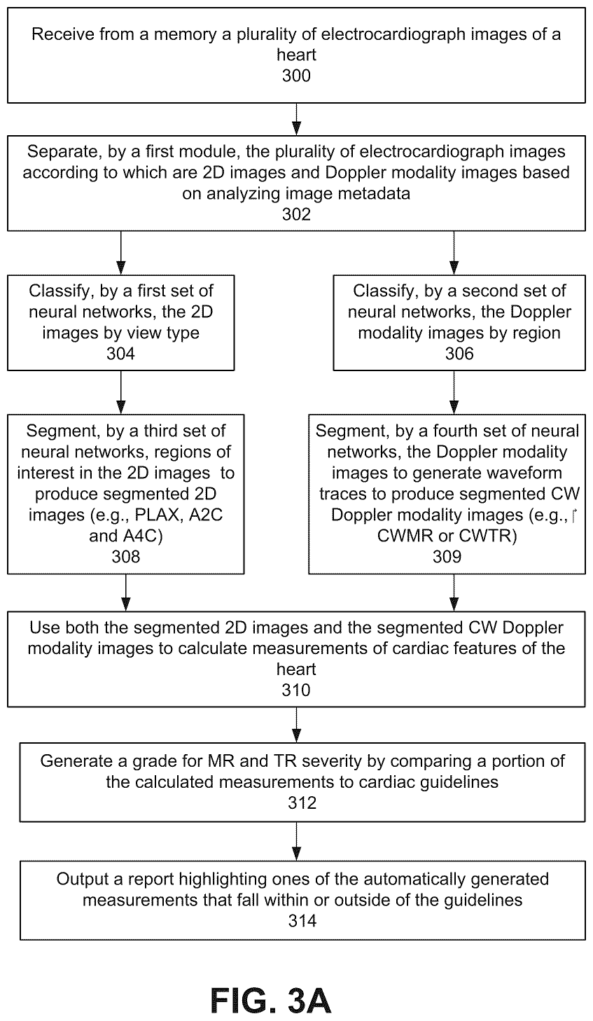

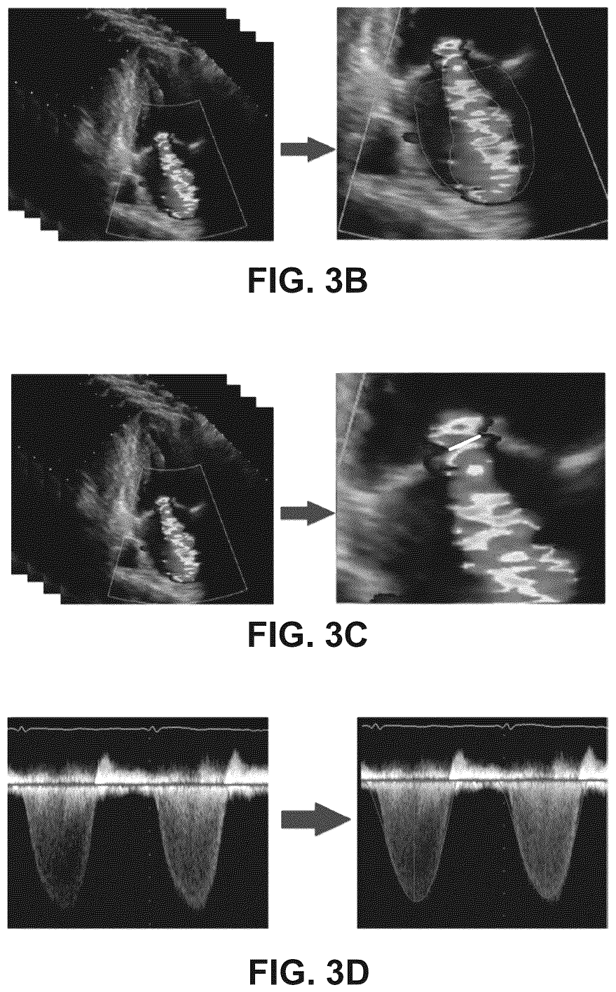

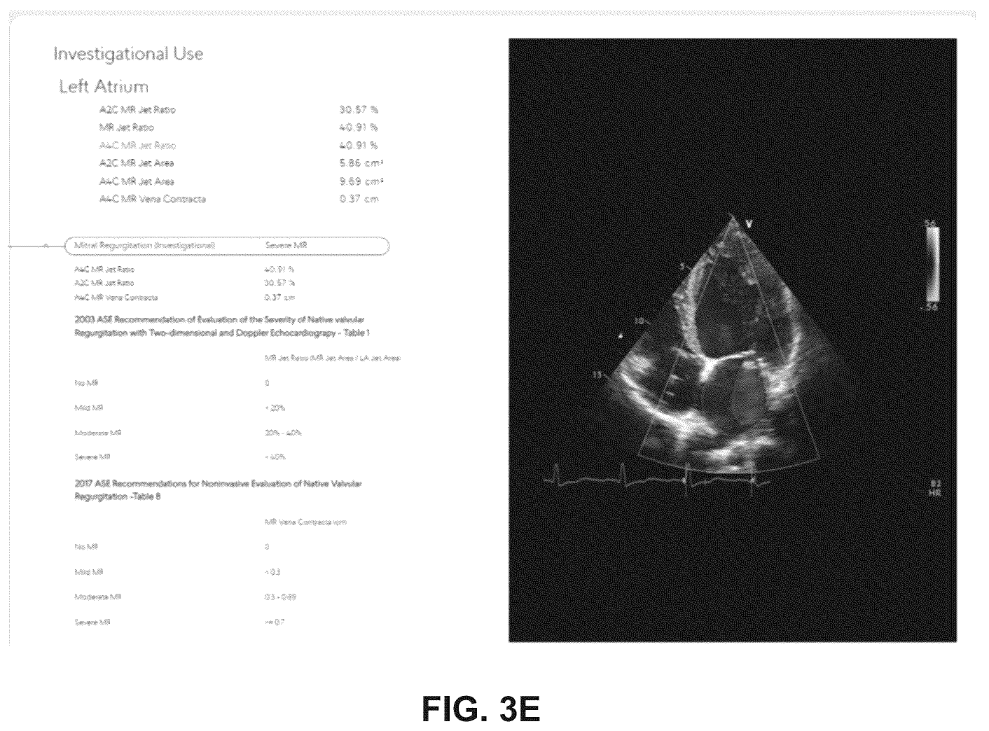

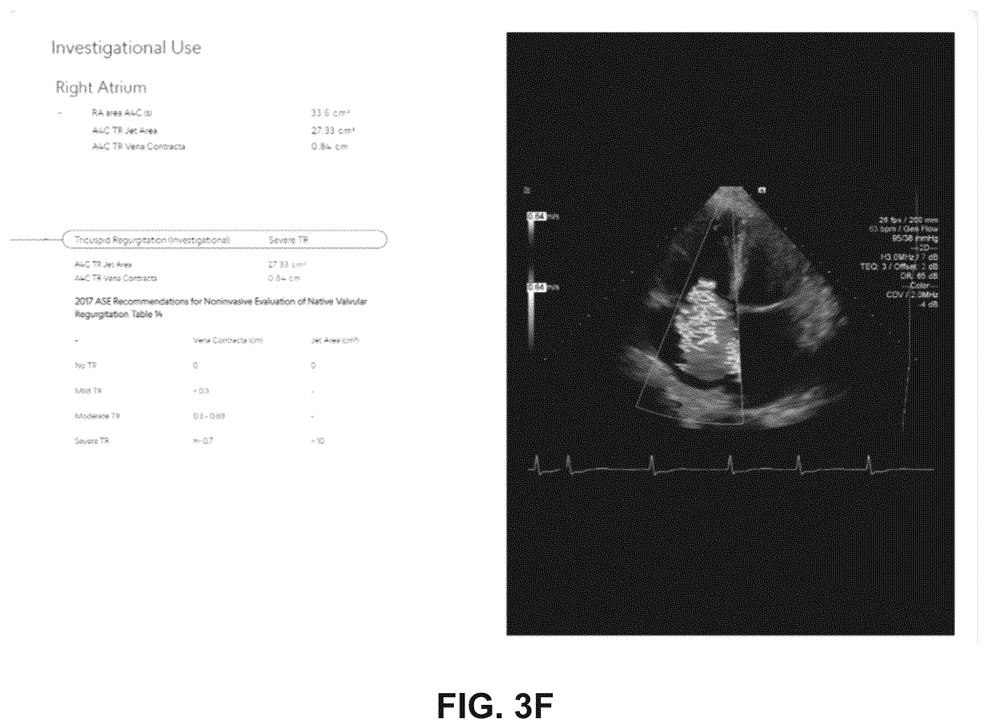

FIG. 3A is a flow diagram illustrating one embodiment of a process for performed by the echo workflow engine 12 to automatically recognize and analyze both 2D and Doppler modality echo images to perform automated measurements and measurements and grading of MR and TR severity. For evaluation of MR, the 2D image view type used are parasternal long axis (PLAX) and apical 2- and 4-chamber (A2C, A4C). For TR, only apical 2 chamber views are used. For MR, the Doppler modality view types used are continuous wave of the mitral valve (CWMR) and continuous wave of the tricuspid valve for (CWTR). The process occurs once the echo workflow engine 12 is trained and placed in analysis mode.

The process may begin by the echo workflow engine 12 receiving from a memory a plurality of echocardiogram images taken by an ultrasound device of a heart (block 300). In one embodiment, the patient study may include 70-90 images and videos.

A first module of the echo workflow engine 12 may be used to operate as a filter to separate the plurality of echocardiogram images according to 2D images and Doppler modality images based on analyzing image metadata (block 302). The first module analyzes the DICOM tags, or metadata, incorporated in the image, and runs an algorithm based upon the tag information to distinguish between 2D and modality images, and then separate the modality images into either pulse wave, continuous wave, PWTDI or m-mode groupings. A second module of the echo workflow engine 12 may perform color flow analysis on extracted pixel data using a combination of analyzing both DICOM tags/metadata and color content within the images, to separate views that contain color from those that do not. A third module may anonymize the data by removing metatags that contain personal information and cropping the images to exclude any identifying information. A fourth module may extract the pixel data from the images and converts the pixel data to numpy arrays for further processing. These functions may be performed by a lesser number or a greater number of models in other embodiments.

Because sonographers do not label the view types in the echo images, one or more of the neural networks are used to classify the echo images by view type. In one embodiment, the first set of one or more neural networks is used by the echo workflow engine 12 to classify the 2D images by view type (block 304); and the second set of neural networks is used by the echo workflow engine 12 to classify, the Doppler modality images by Doppler modality region (block 306). As shown, the processing of 2D images is separate from the processing of Doppler modality images. In one embodiment, the first and second neural networks may be implemented using the set of classification convolutional neural networks (CNNs) 200A. In one specific embodiment, a five class CNN may be used to classify the 2D images by view type and an 11 class CNN may be used to classify the Doppler modality images by Doppler modality region. In an alternative embodiment, the first and second neural networks may be combined into one neural network. In one embodiment, the classification of the echo images by view type and regions by the neural networks can be based on a majority voting scheme to determine the optimal answer. For example, a video can be divided into still image frames, and the workflow engine generates for each image frame a classification label, where the label constitutes a vote, and a particular one of the classification labels receiving the highest number of votes is applied as the classification of the video.

In one embodiment, the echo workflow engine 12 is trained to classify many different view types. For example, the echo workflow engine 12 may be configured to classify any number of different view types including: parasternal long axis (PLAX), apical 2-, 3-, and 4-chamber (A2C, A3C, and A4C), A4C plus pulse wave of the mitral valve, A4C plus pulse wave tissue Doppler on the septal side, A4C plus pulse wave tissue Doppler on the lateral side, A4C plus pulse wave tissue Doppler on the tricuspid side, A5C plus continuous wave of the aortic valve, A4C+Mmode (TrV), A5C, and by different Continuous Wave (CW) regions and PW LVOT (Pulsed Wave Left Ventricular Outflow Tract Doppler). According to one aspect of the disclosed embodiments, the CW regions may be used to measure a parameter, referred to herein as Continuous Wave Doppler Density (CWDD), in which masks are generated for CW waveforms to generate average pixels values within a given waveform from with MR and TR severity is then graded, as explained further below.

Based on the classified images, a third set of neural networks is used by the echo workflow engine 12 to segment regions of interest (e.g., cardiac chambers) in the 2D images to produce annotated or segmented 2D images, including PLAX segmented images and A2C and A4C segmented images (block 308). A fourth set of neural networks is used by the echo workflow engine 12 on each Doppler modality region to generate waveform traces for the Doppler modality images to generate annotated or segmented CW Doppler modality images, including CWMR or CWTR segmented images (block 309). The process of segmentation includes determining locations where each of the cardiac chambers begin and end to generate outlines of structures of the heart (e.g., cardiac chambers) depicted in each image and/or video. Segmentation can also be used to trace the outline of the waveform depicting the velocity of blood flow in a Doppler modality.

In one embodiment, the third and fourth sets of neural networks may be referred to as segmentation neural networks and may comprise the set of segmentation CNNs 200B and 200C. The choice of segmentation CNN used is determined by the view type of the image, which makes the prior correct classification of view type a crucial step. In a further embodiment, once regions of interest are segmented, a separate neural network can be used to smooth outlines of the segmentations.

In one embodiment, the segmentation CNNs may be trained from hand-labeled real images or artificial images generated by general adversarial networks (GANs). In one embodiment, different segmentation CNNs may be trained for semantic segmentation of the PLAX, A2C/A4C, and CW images. From the segmentation of each wave, relevant measurements can be calculated.

Using both the segmented 2D PLAX, A2C and A4C segmented images and the segmented CW Doppler modality segmented images, the echo workflow engine 12 calculates measurements of cardiac features of the heart (block 310). In one embodiment, measurements may of cardiac features be taken of the left side, the right side of the heart, or both.

According to one aspect of the disclosed embodiments, to grade severity of MR, the echo workflow engine 12 performs automated measurement and calculations for the following combination of MR parameters: i) mitral valve vena contracta (VC) width comprising a mean of the measurements in the PLAX segmented images, the A2C segmented images, and the A4C segmented images; ii) mitral regurgitation jet area to left atrial area ratio (JAR); and iii) MR CW Doppler Density (CWDD).

Similarly, to grade severity of TR, the echo workflow engine 12 performs automated measurement and calculations for the following TR parameters: i) tricuspid valve vena contracta (VC) width in the A4C segmented images; ii) tricuspid regurgitation jet area (JA) in the A4C segmented images; and iii) TR CW Doppler Density (CWDD).

FIG. 3B illustrates an example A2C image on which MR jet area ratio (JAR) segmentation is performed. FIG. 3C illustrates an example A2C image on which MR vena contracta (VC) segment annotation (straight line) is performed.

FIG. 3C illustrates an example CWMR image on which CW Doppler Density (CWDD) detection is performed using a pixel density mask segment annotation. According to a further aspect of the disclosed embodiments, the echo workflow engine 12 calculates the MR CWDD parameter and the TR CWDD parameter for a given CW image by generating a mask; assigning a numerical values in grayscale to pixels comprising the mask; normalizing the numerical values to a brightness of the entire CW waveform image to generate normalized brightness values; and calculating the CWDD parameter as an average of the normalized brightness values.

The echo workflow engine 12 then generates a grade of MR or TR severity by comparing a portion of the calculated measurements to cardiac guidelines (block 312). The echo workflow engine 12 may classify the MR or TR severity (none to severe) by determining a grade for each of the MR or TR parameters. The highest grade for each of the three MR or TR parameters is used to determine a final MR or TR severity grade. In one embodiment, MR or TR severity may be categorized as non-significant (none/trivial/mild-nsMR-) or significant (moderate/severe-sMR-) based on the measured VC, JAR or JA values. In one embodiment, VC, JAR or JA values may be compared to international guidelines, but CWDD is a novel measurement so there is no comparable guideline measurement.

Example cardiac guidelines that may be used as a reference may internation guidelines such as: “AHA/ACC Guideline For The Management Of Patients With Valvular Heart Disease”, a report of the American College of Cardiology/American Heart Association Task Force on Practice Guidelines (Circulation 2014); and “Joint Task Force on the Management of Valvular Heart Disease of the European Society of Cardiology (ESC),” European Association for Cardio-Thoracic Surgery (EACTS), Guidelines on the management of valvular heart disease (version 2012).

The echo workflow engine 12 further outputs at least one report to a user showing ones of the calculated measurements that fall within or outside of the international guidelines (block 314). In one embodiment, two reports are generated and output: the first report is a list of the calculated values for each measurement with the highest confidence as determined by a rules based engine, highlighting values among the calculated measurements that fall outside of the international guidelines; and the second report is a comprehensive list of all measurements calculated on every image frame of every video, in every view, generating large volumes of data. All report data and extracted pixel data may be stored in a structured database to enable machine learning and predictive analytics on images that previously lacked the quantification and labelling necessary for such analysis. The structured database may be exported to a cloud-based server or may remain on premises (e.g., of the lab owning the images) and can be connected to remotely. By connecting these data sources into a single network, the disclosed embodiments can progressively train disease prediction algorithms across multiple network nodes and validate the algorithms in distinct patient cohorts. In one embodiment, the reports may be electronically displayed to a doctor and/or a patient on a display of an electronic device and/or as a paper report. In some embodiments, the electronic reports may be editable by the user per rule or role-based permissions, e.g., a cardiologist may be allowed to modify the report, but a patient may have only view privileges.

FIGS. 3E and 3F are diagrams illustrating an example reports of MR and TR severity, respectively.

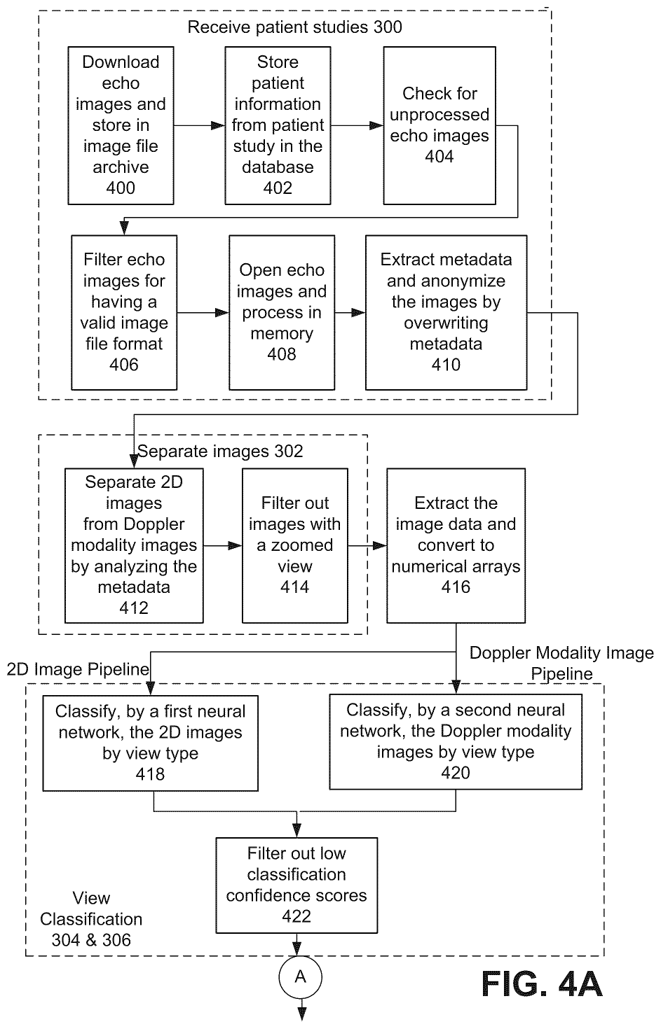

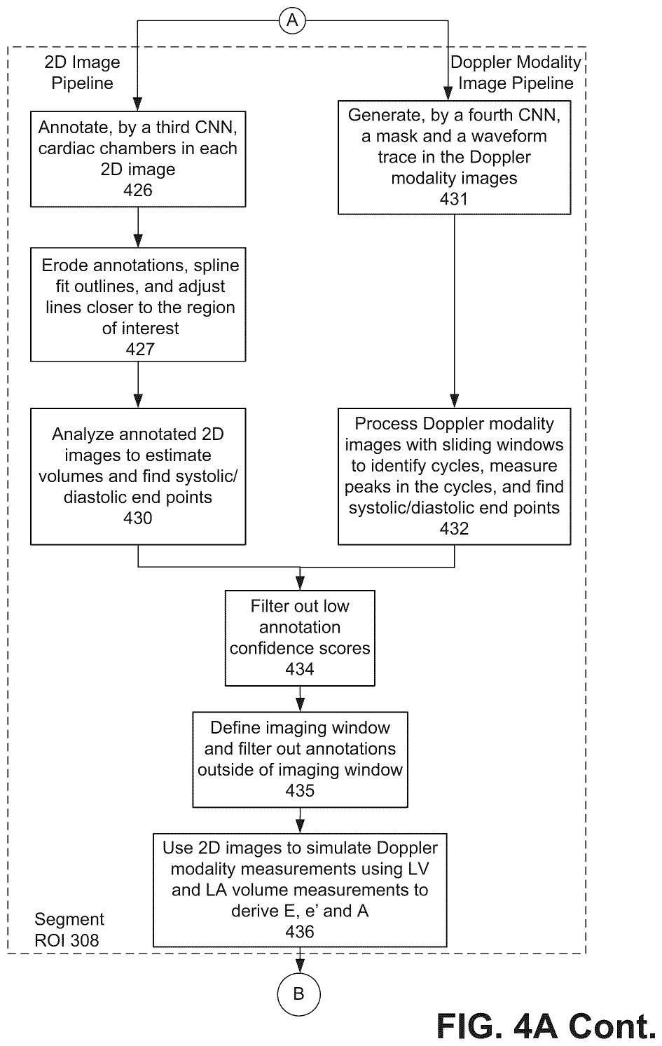

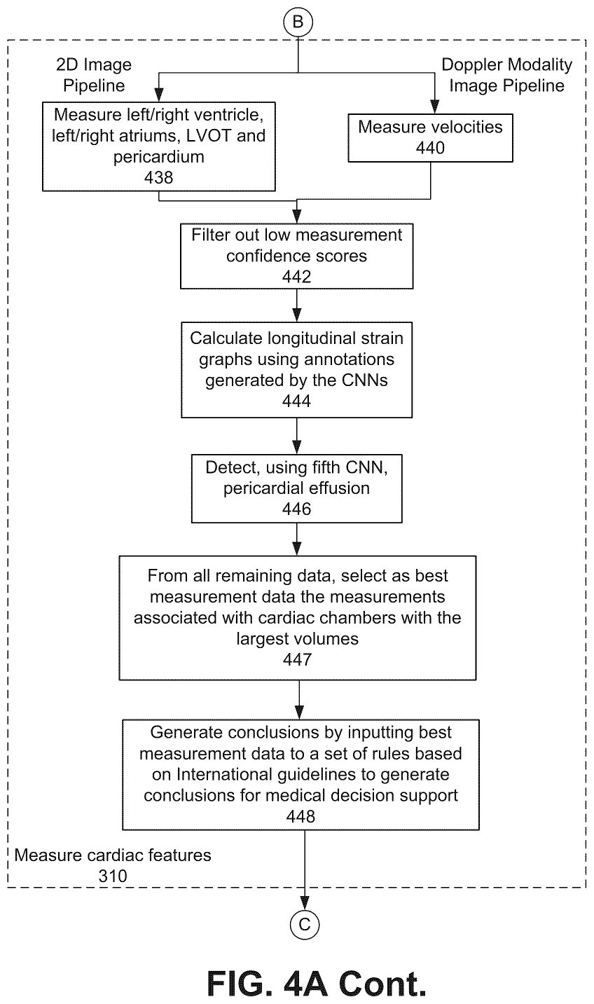

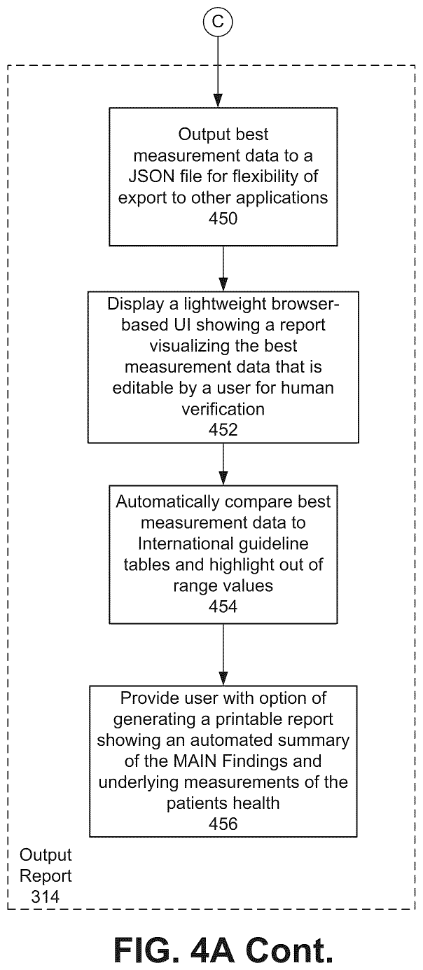

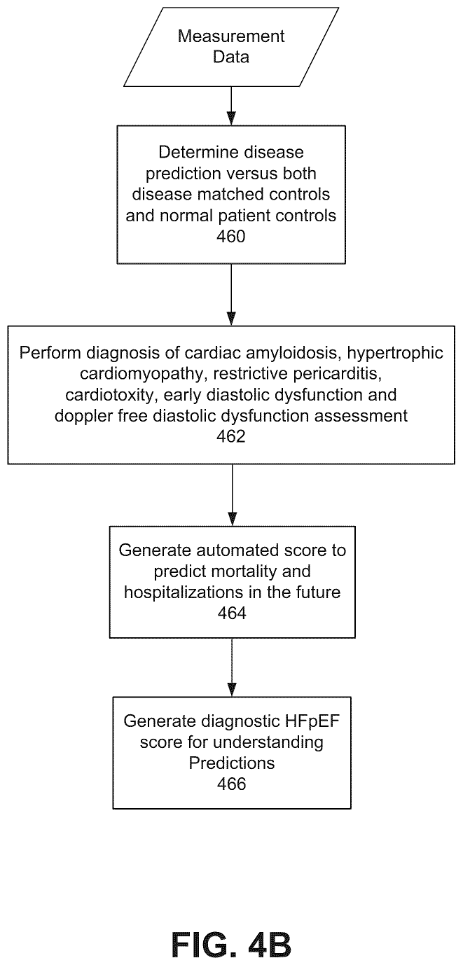

FIG. 4A is a flow diagram illustrating further details of the process for automatically recognizing and analyze both 2D and Doppler modality echo images to perform automated measurements and the diagnosis, prediction and prognosis of heart disease according to one embodiment.

The process may begin with receiving one or more patient studies (FIG. 3 block 300), which comprises blocks 400-4010. In one embodiment, echo images from each of the patient studies are automatically downloaded into the image file archive 18 (block 400). The echo images may be received from a directly from local or remote storage source of the computer 14. The local storage sources may include internal/external storage of the computer 14 including removable storage devices. The remote storage sources may include the ultrasound imaging device 24, the DICOM servers 30, the network file share devices 32, the echo workstations 34, and/or the cloud storage services 36 (see FIG. 1). In one embodiment, the echo workflow engine 12 includes functions for operating as a picture archiving and communication server (PACS), which is capable of handling images from multiple modalities (source machine types, one of which is the ultrasound imaging device 24). The echo workflow engine 12 uses PACS to download and store the echo images into the image file archive 18 and provides the echo workflow engine 12 with access to the echo images during the automated workflow. The format for PACS image storage and transfer is DICOM (Digital Imaging and Communications in Medicine).

Patient information from each of the patient studies is extracted and stored in the database 16 (block 402). Non-image patient data may include metadata embedded within the DICOM images and/or scanned documents, which may be incorporated using consumer industry standard formats such as PDF (Portable Document Format), once encapsulated in DICOM. In one embodiment, received patient studies are placed in a processing queue for future processing and during the processing of each patient study, the echo workflow engine 12 queues and checks for unprocessed echo images (block 404). The echo workflow engine 12 monitors the status of patient studies, and keeps track of them in a queue to determine which have been processed and which are still pending. In one embodiment, prioritization of the patient studies in the queue may be configured by a user. For example, the patient studies may be prioritized in the queue for processing according to the date of the echo exam, the time of receipt of the patient study or by estimated severity of the patient's heart disease.

Any unprocessed echo images are then filtered for having a valid DICOM image format and non DICOM files in an echo study are discarded (block 406). In one embodiment, the echo images are filtered for having a particular type of format, for example, a valid DICOM file format, and any other file formats may be ignored. Filtering the echo images for having a valid image file format enhances the reliability of the echo workflow engine 12 by rejecting invalid DICOM images for processing.

Any unprocessed valid echo images are then opened and processed in the memory of the computer 14 (block 408). Opening of the echo images for the patient study in memory of the computer 14 is done to enhance processing speed by echo workflow engine 12. This is in contrast to an approach of opening the echo files as sub-processes, saving the echo files to disk, and then reopening each echo image during processing, which could significantly slow processing speed.

The echo workflow engine 12 then extracts and stores the metadata from the echo images and then anonymizes the images by blacking out the images and overwriting the metadata in order to protect patient data privacy by covering personal information written on the image (block 410). As an example, DICOM formatted image files include metadata referred to as DICOM tags that may be used to store a wide variety of information such as patient information, Doctor information, ultrasound manufacture information, study information, and so on. In one embodiment, the extracted metadata may be stored in the database 16 and the metadata in image files is overwritten for privacy.

After receipt and processing of the patient studies, the echo workflow engine 12 separates 2D images from Doppler modality images so the two different image types can be processed by different pipeline flows, described below. In one embodiment, the separating of the images (FIG. 3, block 302) may comprise blocks 412-414. First, the 2D images are separated from the Doppler modality images by analyzing the metadata (block 412).

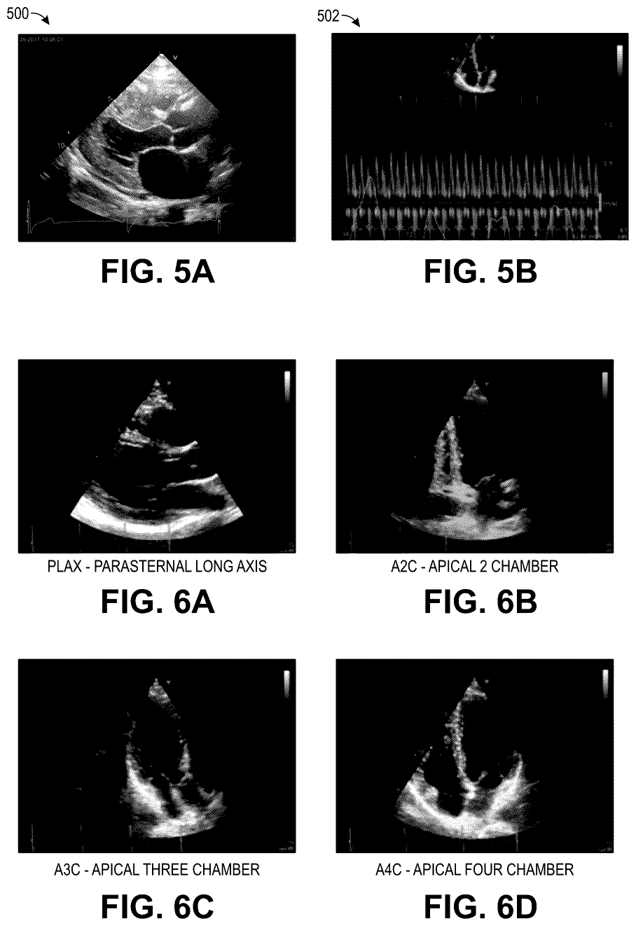

FIGS. 5A and 5B are diagrams illustrating an example 2D echo image 500 and an example Doppler modality image 502 including a waveform, respectively. The echo workflow engine 12 may determine the image type by examining metadata/DICOM tags. In one embodiment, information within the DICOM tags may be extracted in order to group the images into one of the following four classes: 2D only, pulsed-wave (PW), continuous wave (CW), and m-mode. Similarly, the transducer frequency of the ultrasound imaging device 24 in the metadata may be used to further separate some of the PW images into a fifth class: pulsed-wave tissue doppler imaging (PWTDI).

Referring again to FIG. 4A, the echo workflow engine 12 may also filter out images with a zoomed view, which may also be determined by analyzing the metadata (block 414). Any of the echo images that has been zoomed during image capture are not processed through the pipeline because when zooming, useful information is necessarily left out of the image, meaning the original image would have to be referenced for the missing data, which is a duplication of effort that slows processing speed. Accordingly, rather than potentially slowing the process in such a manner, the echo workflow engine 12 filters out or discards the zoomed images to save processing time. In an alternative embodiment, filtering out zoomed images in block 414 may be performed prior to separating the images in block 412.

After separating the 2D images from the Doppler modality images, the echo workflow engine 12 extracts and converts the image data from each echo image into numerical arrays (block 416). For the echo images that are 2D only, the pixel data comprises a series of image frames played in sequence to create a video. Because the image frames are unlabeled, the view angle needs to be determined. For the Doppler modality images that include waveform modalities, there are two images in the DICOM file that may be used for subsequent view identification, a waveform image and an echo image of the heart. The pixel data is extracted from the DICOM file and tags in the DICOM file determine the coordinates to crop the images. The cropped pixel data is stored in numerical arrays for further processing. In one embodiment, blocks 412, 414 and 416 may correspond to the separating images block 302 of FIG. 3.

After separating images, the echo workflow engine 12 attempts to classify each of the echo images by view type. In one embodiment, view classification (FIG. 3 blocks 304 and 306) corresponds to blocks 418-422.

According to the disclosed embodiments, the echo workflow engine 12 attempts to classify each of the echo images by view type by utilizing parallel pipeline flows. The parallel pipeline includes a 2D image pipeline and a Doppler modality image pipeline. The 2D pipeline flow begins by classifying, by a first CNN, the 2D images by view type (block 418), corresponding to block 304 from FIG. 3. The Doppler modality image pipeline flow begins by classifying, by a second CNN, the Doppler modality images by view type (block 420), corresponding to block 306 from FIG. 3.

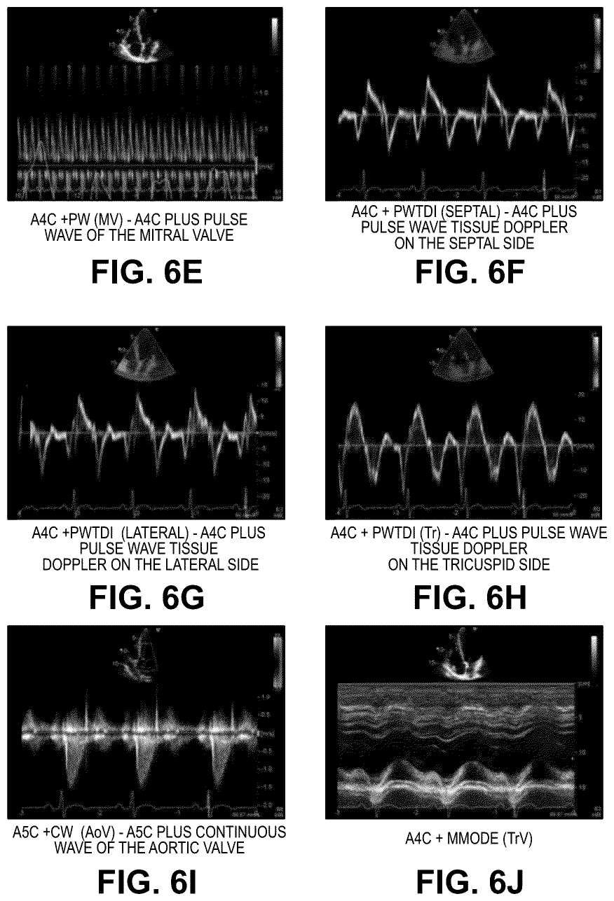

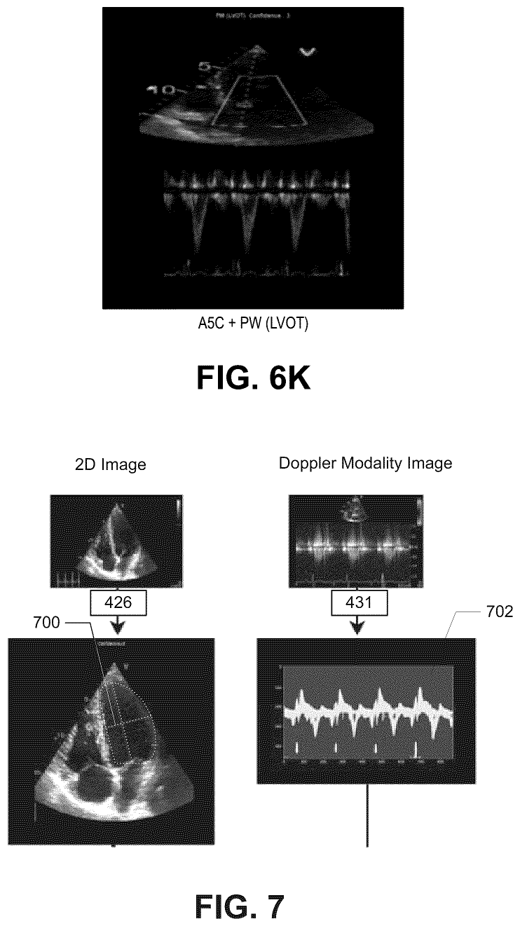

FIGS. 6A-6K are diagrams illustrating some example view types automatically classified by the echo workflow engine 12. As stated previously, example view types may include parasternal long axis (PLAX), apical 2-, 3-, and 4-chamber (A2C, A3C, and A4C), A4C plus pulse wave of the mitral valve, A4C plus pulse wave tissue Doppler on the septal side, A4C plus pulse wave tissue Doppler on the lateral side, A4C plus pulse wave tissue Doppler on the tricuspid side, A5C plus continuous wave of the aortic valve, A4C+Mmode (TrV), A5C+PW (LVOT).

Referring again to FIG. 4A, in one embodiment, 2D image classification is performed as follows. If the DICOM file contains video frames from a 2D view, only a small subset of the video frames are analyzed to determine 2D view classification for more efficient processing. In one embodiment, the subset of the video frames may range approximately 8-12 video frames is, but preferably 10 frames are input into one of the CNNs 200A trained for 2D to determine the actual view. In an alternative embodiment, a subset of video frames may be randomly selected from the video file. In one embodiment, the CNNs 200A classify each of the analyzed video frames as one of: A2C, A3C, A4C, A5C, PLAX Modified, PLAX, PSAX AoV level, PSAX Mid-level, Subcostal Ao, Subcostal Hep vein, Subcostal IVC, Subcostal LAX, Subcostal SAX, Suprasternal and Other.

Doppler modality images comprise two images, an echocardiogram image of the heart and a corresponding waveform, both of which are extracted from the echo file for image processing. In one embodiment, Doppler modality image classification of continuous wave (CW), pulsed-wave (PW), and M-mode images is performed as follows. If the DICOM file contains a waveform modality (CW, PW, PWTDI, M-mode), the two extracted images are input to one of the CNNs 200A trained for CW, PW, PWTDI and M-mode view classification to further classify the echo images as one of: CW (AoV), CW (TrV), CW Other, PW (LVOT), PW (MV), PW Other, PWTDI (lateral), PWTDI (septal), PWTDI (tricuspid), M-mode (TrV) and M-mode Other.

There are many more potential classifications available for modalities, but the present embodiments strategically select the classes above, while grouping the remaining potential classes into “Other”, in order to maximize processing efficiency, while identifying the most clinically important images for further processing and quantification. Customization of the CNNs 200A occurs in the desired number of layers used and the quantity of filters within each layer. During the training phase, the correct size of the CNNs may be determined through repeated training and adjustments until optimal performance levels are reached.

During view classification, the echo workflow engine 12 maintains classification confidence scores that indicate a confidence level that the view classifications are correct. The echo workflow engine 12 filters out the echo images having classification confidence scores that fail to meet a threshold, i.e., low classification confidence scores (block 422). Multiple algorithms may be employed to derive classification confidence scores depending upon the view in question. Anomalies detected in cardiac structure annotations, image quality, cardiac cycles detected, and the presence of image artifacts may all serve to decrease the classification confidence score and discard an echo image out of the automated echo workflow.

With respect to the confidence scores, the echo workflow engine 12 generates and analyzes several different types of confidence scores at different stages of processing, including classification, annotation, and measurements (e.g., blocks 422, 434 and 442). For example, poor quality annotations or classifications, which may be due to substandard image quality, are filtered out by filtering the classification confidence scores. In another example, in a patient study the same view may be acquired more than once, in which case the best measurements are chosen by filtering out low measurement confidence scores as described further below in block 442. Any data having a confidence score that meets a predetermined threshold continues through the workflow. Should there be a duplication of measurements both with high confidence, the most clinically relevant measurement may be chosen.

Next the echo workflow engine 12 performs image segmentation to define regions of interest (ROI). In computer vision, image segmentation is the process of partitioning a digital image into multiple segments (sets of pixels) to locate and boundaries (lines, curves, and the like) of objects. Typically, annotations are a series of boundary lines overlaying overlaid on the image to highlight segment boundaries/edges. In one embodiment, the segmentation to define ROI (FIG. 3 block 308) corresponds to blocks 426-436.

In one embodiment, the 2D image pipeline annotates, by a third CNN, regions of interests, such as cardiac chambers in the 2D images, to produce annotated 2D images (block 426). An annotation post process then erodes the annotations to reduce their dimensions, spline fits outlines of cardiac structures and adjusts locations of the boundary lines closer to the region of interest (ROIs) (block 427). The 2D image pipeline continues with analyzing the ROIs (e.g., cardiac chambers) in the annotated 2D images to estimate volumes and determine key points in the cardiac cycle by finding systolic/diastolic end points (block 430). For 2D only views, measurements are taken at the systolic or diastolic phase of the cardiac cycle, i.e. when the left ventricle reaches the smallest volume (systole) or the largest volume (diastole). From the 2D video images, it must be determined which end points are systolic and which are diastolic based on the size of the estimated volumes of the left ventricle. For example, a significantly large left ventricle may indicate a dystonic end point, while a significantly small volume may indicate a systolic end point. Every video frame is annotated and the volume of the left ventricle is calculated throughout the whole cardiac cycle. The frames with minimum and maximum volumes are detected with a peak detection algorithm.

The Doppler modality pipeline analyzes the Doppler modality images and generates, by a fourth CNN, a mask and a waveform trace in the Doppler modality images to produce annotated Doppler modality images (block 431).

FIG. 7 is a diagram illustrating an example 2D image segmented in block 426 to produce annotations 700 indicating cardiac chambers, and an example Doppler modality image segmented in block 431 to produce a mask and waveform trace 702.

In one embodiment, the third and fourth CNNs may correspond to segmentation CNNs 200B. In one embodiment, each of the CNNs 200B used to segment the 2D images and Doppler modality images may be implemented as U-Net CNN, which is convolutional neural network developed for biomedical image segmentation. Multiple U-Nets may be used. For example, for 2D images, a first U-Net CNN can be trained to annotate ventricles and atria of the heart from the A2C, A3C, A4C views. A second U-net CNN can be trained to annotate the chambers in the PLAX views. For M-mode views, a third U-Net CNN can be trained to segment the waveform, remove small pieces of the segments to find likely candidates for the region of interest, and then reconnect the segments to provide a full trace of the movement of the mitral valve. For CW views, a fourth U-net CNN can be trained to annotate and trace blood flow. For PW views, a fifth U-net CNN trained to annotate and trace the blood flow. For PWTDI views, a sixth U-net CNN can be trained to annotate and trace movement of the tissues structures (lateral/septal/tricuspid valve).

Referring again to FIG. 4A, the Doppler modality pipeline continues by processing the annotated Doppler modality images with a sliding window to identify cycles, peaks are measured in the cycles, and key points in the cardiac cycle are determined by finding systolic/diastolic end points (block 432). Typically, a Doppler modality video may capture three heart cycles and the sliding window is adjusted in size to block out two of the cycles so that only one selected cycle is analyzed. Within the selected cycle, the sliding window is used to identify cycles, peaks are measured in the cycles, and key points in the cardiac cycle are determined by finding systolic/diastolic end points.

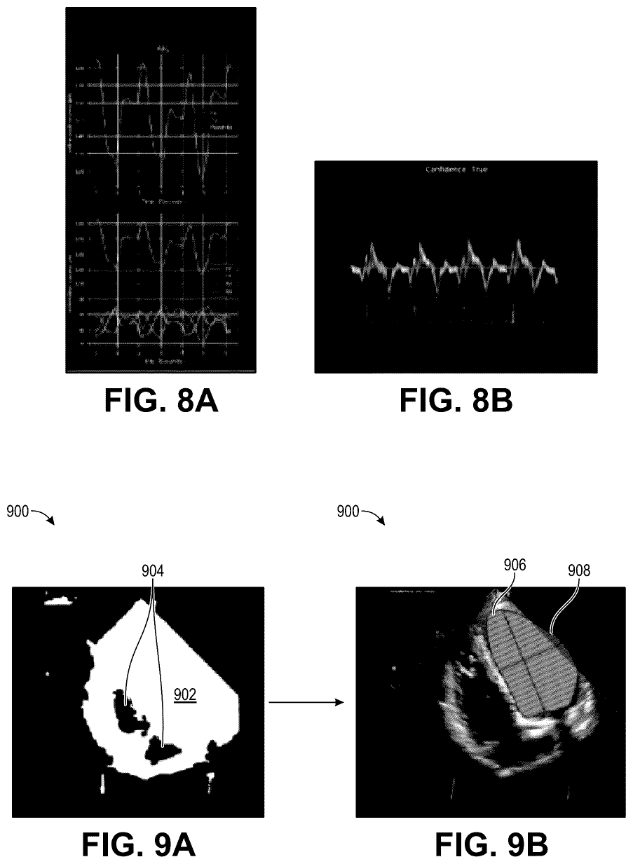

FIGS. 8A and 8B are diagrams illustrating examples of finding systolic/diastolic end points in the cardiac cycle, for both 2D and Doppler modalities, respectively, which are key points in order to take accurate cardiac measurements.

Referring again to FIG. 4A, in one embodiment, the echo workflow engine 12 maintains annotation confidence scores corresponding to the estimated volumes, systolic/diastolic end points, identified cycles and measured peaks. The echo workflow engine 12 filters out annotated images having annotation confidence scores that fail to meet a threshold, i.e., low annotated confidence scores (block 434). Examples of low confidence annotations may include annotated images having one or more of: excessively small areas/volumes, sudden width changes, out of proportion sectors, partial heart cycles, and insufficient heart cycles.

After images having low annotation confidence scores are filtered out, the echo workflow engine 12 defines an imaging window for each image and filters out annotations that lie outside of the imaging window (block 435).

FIGS. 9A and 9B are diagrams illustrating processing of an imaging window in a 2D echo image 900. In FIG. 9A, a shaded ROI 902 is shown having unshaded regions or holes 904 therein. Open CV morphology transformations are used to fill the holes 904 inside the ROI, as shown in In FIG. 9B. Thereafter, a Hough line transformation may be used to find an imaging window border 906. As is well known, a Hough transform is a feature extraction technique used in digital image processing to find imperfect instances of objects within a certain class of shapes. After an imaging window border is found, a pixel account of annotations beyond the imaging window border is made. Annotations 908 with a significant number of pixels outside the border of the imaging window are then discarded.

Referring again to FIG. 4A, in the handheld configuration for the automated clinical workflow system 10C (See FIG. 1B), the patient studies may not include Doppler modality images. According to a further aspect, the disclosed embodiments accommodate for such handheld configurations by using the 2D images to simulate Doppler modality measurements by using Left Ventricular (LV) and Left Atrial (LA) volume measurements to derive E, e′ and A (e.g., early and late diastolic transmittal flow and early/mean diastolic tissue velocity) measurements (block 436). In one embodiment, simulating the Doppler modality measurements may be optional and may be invoked based on a software setting indicating the presence of a handheld configuration and/or absence of Doppler modality images for the current patient study.

Referring again to FIG. 4A, in one embodiment, once cardiac features are annotated during segmentation, the cardiac features are then measured during a measurement process (block 310 of FIG. 3), which in one embodiment may comprise block 438-448. The process of measuring cardiac features may begin by quantifying a plurality of measurements using the annotations. First, the 2D pipeline measures the 2D images left/right ventricle, left/right atriums, left ventricular outflow (LVOT) and pericardium (block 438). For the Doppler modality images, the Doppler modality image pipeline measures blood flow velocities (block 440).

More specifically, for A2C, A3C, A4C, and A5C image views, volumetric measurements of chamber size are conducted on the systolic and diastolic frames of the video, and image processing techniques mimic a trained clinician at measuring the volume using the method of disks (MOD). For 2D Plax, PSAX (mid-level), PSAX (AoV level), Subcostal, Suprasternal and IVC image views, linear measurements of chamber size and inter-chamber distances are conducted on the systolic and diastolic frames of the video using image processing techniques to mimic the trained clinician. For M-mode image views, from the annotated segments of the movement of the tricuspid valve, a center line is extracted and smoothed, and then the peaks and valleys are measured in order to determine the minimum and maximum deviations over the cardiac cycle. For PW image views, from the annotations of the blood flow, a mask is created to isolate parts of the waveform. A sliding window is then run across the trace to identify one full heart cycle, in combination with heart rate data from the DICOM tags, to use as a template. This template is then used to identify all other heart cycles in the image. Peak detection is then performed on each cycle and then run through an algorithm to identify which part of the heart cycle each peak represents. For CW image views, from the annotations of the trace of the blood flow, curve fitting is performed on the annotation to then quantify the desired measurements. For PWTDI image views, from the annotations of the movement of the tissue, a mask is created to isolate parts of the waveform. A sliding window is then run across the trace to identify one full heart cycle, in combination with heart rate data from the DICOM tags, to use as a template. This template is then used to identify all other heart cycles in the image. Peak detection is then performed on each cycle and then run through an algorithm to identify which part of the heart cycle each peak represents.

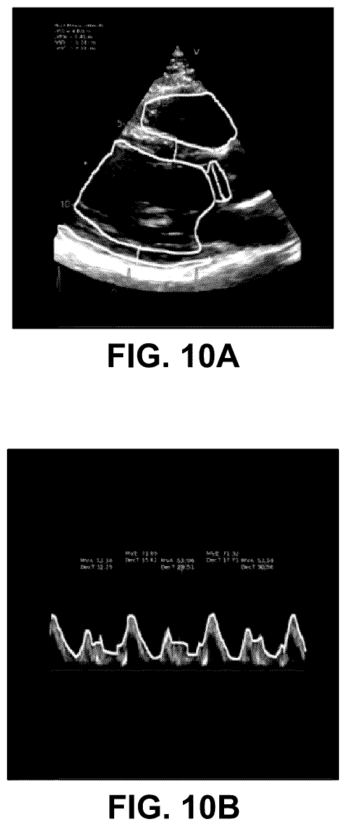

FIGS. 10A and 10B are diagrams graphically illustrating structural measurements automatically generated from annotations of cardiac chambers in a 2D image, and velocity measurements automatically generated from annotations of waveforms in a Doppler modality, respectively.

The measurement table below list the measurements that may be compiled by the echo workflow engine 12 according to one embodiment.

Referring again to FIG. 4A, in one embodiment, the echo workflow engine 12 maintains measurement confidence scores corresponding to the measured left/right ventricles, left/right atriums, LVOTs, pericardium and measured velocities. The echo workflow engine 12 filters out echo images having measurement confidence scores that fail to meet a threshold, i.e., low measurement confidence scores (block 442).

Measurement of cardiac features continues with calculating longitudinal strain graphs using the annotations generated by the CNNs (block 444). Thereafter, a fifth CNN is optionally used to detect pericardial effusion 446.

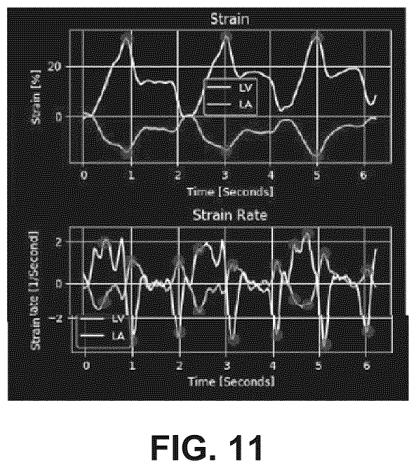

FIG. 11 is a diagram graphically illustrating measurements of global longitudinal strain that were automatically generated from the annotations of cardiac chambers in 2D images.

Referring again to FIG. 4A, for all remaining non-filtered out data, the echo workflow engine 12 selects as best measurement data the measurements associated with cardiac chambers with the largest volumes, and saves with the best measurement data, image location, classification, annotation and other measurement data associated with the best measurements (block 447).

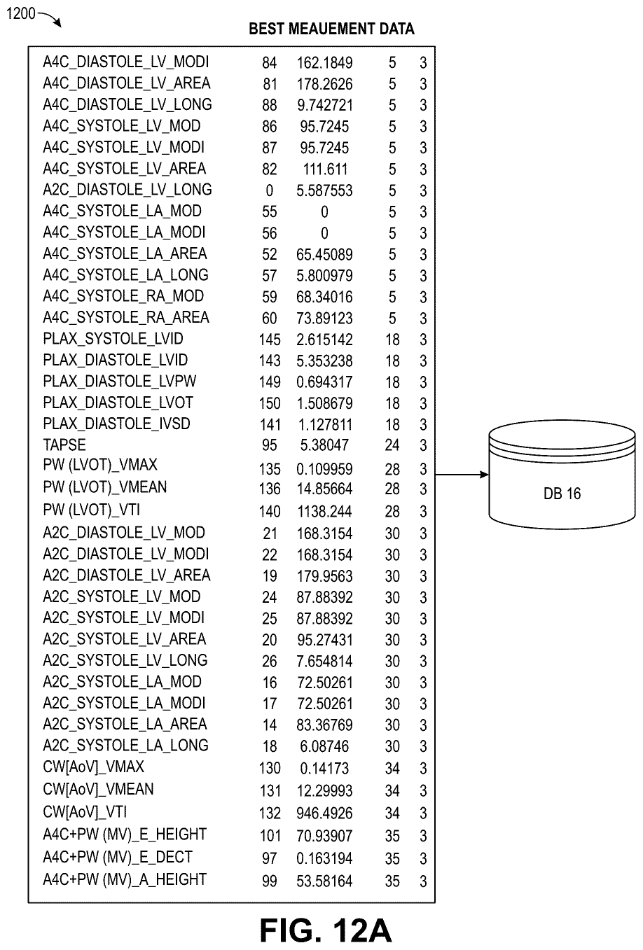

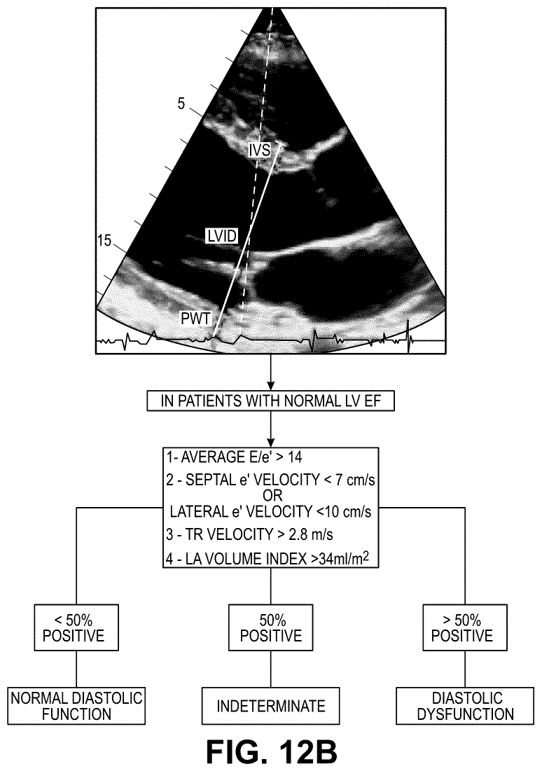

FIG. 12A is a diagram graphically illustrating an example set of best measurement data 1200 based on largest volume cardiac chambers and the saving of the best measurement data 1200 to a repository, such as database 16 of FIG. 1.

Referring again to FIG. 4A, the echo workflow engine 12 then generates conclusions by inputting the best measurement data 1200 to a set of rules based on international measurement guidelines to generate conclusions for medical decisions support (block 448).

FIG. 12B is a diagram graphically illustrating the input of normal LV EF measurements into a set of rules to determine a conclusion that the patient has normal diastolic function, diastolic dysfunction, or indeterminate.

Referring again to FIG. 4A, after the conclusions are generated, a report is generated and output (FIG. 3 block 314), which may comprise blocks 450-456. Report generation may begin by the echo workflow engine 12 outputting the best measurement data 1200 to a JSON file for flexibility of export to other applications (block 450).

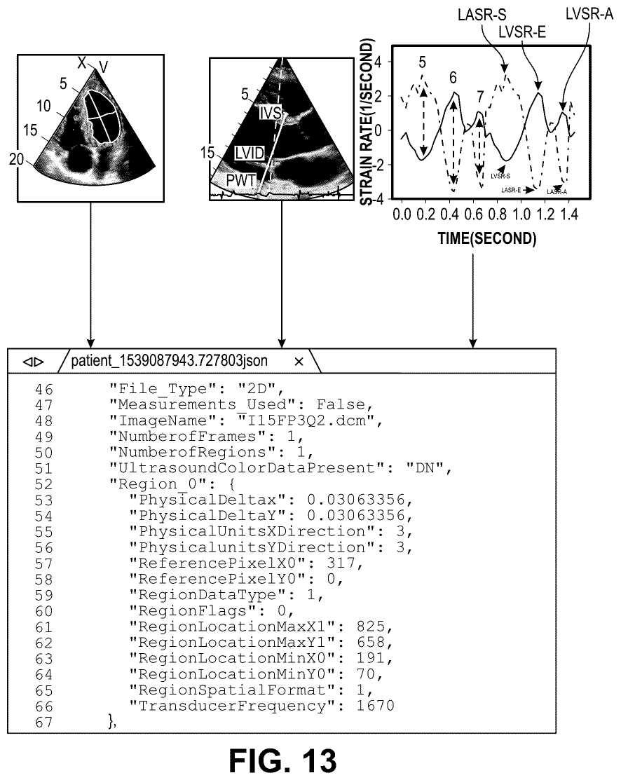

FIG. 13 is a diagram graphically illustrating the output of classification, annotation and measurement data to an example JSON file.

Referring again to FIG. 4A, a lightweight browser-based user interface (UI) is displayed showing a report that visualizes the best measurement data 1200 from the JSON file and that is editable by a user (e.g., doctor/technician) for human verification (block 452). As is well known, a lightweight web browser is a web browser that is optimized to reduce consumption of system resources, particularly to minimize memory footprint, and by sacrificing some of the features of a mainstream web browser. In one embodiment, any edits made to the data are stored in the database 16 and displayed in the UI.

In order to make clinically relevant suggestion to the user, measurements associated with the best measurement data 1200 are automatically compared to current international guideline values and any out-of-range values are highlighted for the user (block 454).

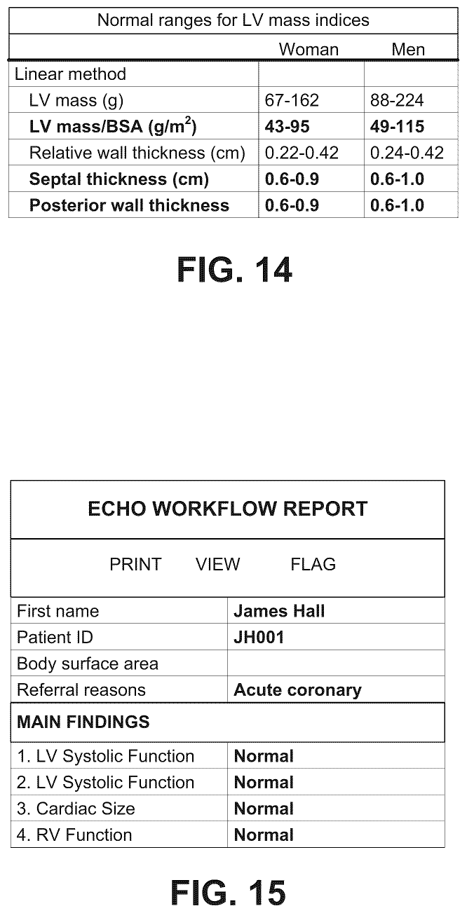

FIG. 14 is a diagram illustrating a portion of an example report showing highlighting values that are outside the range of international guidelines.

Referring again to FIG. 4A, the user is provided with an option of generating a printable report showing an automated summary of Main Findings (i.e., a conclusion reached after examination) and underlining measurements of the patient's health (block 456).

FIG. 15 is a diagram illustrating a portion of an example report of Main Findings that may be printed and/or displayed by the user.

In one embodiment, the automated workflow of the echo workflow engine 12 may end at block 456. However, in further aspects of the disclosed embodiments, the process may continue with advance functions, as described below.

FIG. 4B is a flow diagram illustrating advanced functions of the echo workflow engine. According to this embodiment, the echo workflow engine 12, takes as inputs values of specific measurements that were automatically derived using machine learning (see block 310), and analyzes the specific measurements to determine disease diagnosis/prediction/prognosis versus both disease and matched controls and normal patient controls (block 460). In one embodiment, the echo workflow engine 12 may use the disease predictions to perform diagnosis of any combination of: cardiac amyloidosis, hypertrophic cardiomyopathy, restrictive pericarditis, cardiotoxity, early diastolic dysfunction and Doppler free diastolic dysfunction assessment (block 462). A prognosis in the form of an automated score may then be generated to predict mortality and hospitalizations in the future (block 464).

Echocardiography is key for the diagnosis of heart failure with preserved ejection fraction (HFpEF). However, existing guidelines are mixed in their recommendations for echocardiogram criteria and none of the available guidelines have been validated against gold-standard invasive hemodynamic measurements in HFpEF.

According to one embodiment, the echo workflow engine 12 further generates a diagnostic score for understanding predictions (block 466). Using machine learning, the echo workflow engine 12 validates the diagnostic score against invasively measured pulmonary capillary wedge pressure (PCWP) and determines the prognostic utility of the score in a large HFpEF cohort.

In one embodiment, the echo workflow engine 12, takes as the inputs values of specific measurements that were automatically derived using machine learning workflow, and analyzes the input values using an HFpEF algorithm to compute the HFpEF diagnostic score.

Recognizing that hypertensive heart disease is the most common precursor to HFpEF and has overlapping echocardiogram characteristics with HFpEF, echocardiogram features of 233 patients with HFpEF (LVEF≥50%) was compared to 273 hypertensive controls with normal ejection fraction but no heart failure. An agnostic model was developed using penalized logistic regression model and Classification and Regression Tree (CART) analysis. The association of the derived echocardiogram score with invasively measured PCWP was investigated in a separate cohort of 96 patients. The association of the score with the combined clinical outcomes of cardiovascular mortality of HF hospitalization was investigated in 653 patients with HFpEF from the Americas echocardiogram sub study of the TOPCAT trial.

According to one embodiment, left ventricular ejection fraction (LVEF<60%), peak TR velocity (>2.3 m/s), relative wall thickness (RWT>0.39 mm), interventricular septal thickness (>12.2 mm) and E wave (>1 m/s) are selected as the most parsimonious combination of variables to identify HFpEF from hypertensive controls. A weighted score (range 0-9) based on these 5 echocardiogram variables had a combined area under the curve of 0.9 for identifying HFpEF from hypertensive controls.

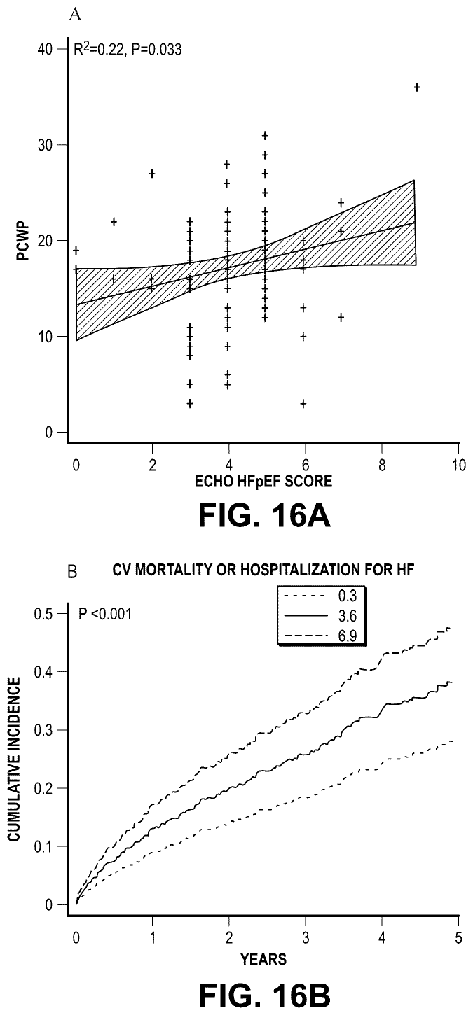

FIGS. 16A and 16B are diagrams showing a graph A plotting PCWP and HFpEF scores, and a graph B plotting CV mortality or hospitalization for HF, respectively. Graph A shows that in the independent cohort, the HFpEF score was significantly associated with PCWP in patients with HFpEF (R²=0.22, P=0.034). Graph B shows that a one-point increase was associated with a 12% increase in risk (hazard ratio [HR] 1.12; 95% CI 1.02-1.23, P=0.015) for the combined outcome after multivariable correction.

According to the disclosed embodiments, the echocardiographic score can distinguish HFpEF from hypertensive controls and is associated with objective measurements of severity and outcomes in HFpEF.

A method and system for implementing a software-based automatic clinical workflow using machine learning that recognizes and analyzes both 2D and Doppler modality Echocardiographic images for grading of mitral valve and tricuspid valve regurgitation, and which can be deployed in workstation or mobile-based ultrasound point-of-care systems has been disclosed. The present invention has been described in accordance with the embodiments shown, and there could be variations to the embodiments, and any variations would be within the spirit and scope of the present invention. For example, the disclosed embodiment can be implemented using hardware, software, a computer readable medium containing program instructions, or a combination thereof. Accordingly, many modifications may be made by one of ordinary skill in the art without departing from the spirit and scope of the appended claims.

We claim:

1. A computer-implemented method for grading of mitral valve regurgitation (MR) or tricuspid valve regurgitation (TR) severity performed by an automated workflow engine executed by at least one processor, the method comprising:
receiving, from a memory, a plurality of echocardiogram images a heart;
separating the plurality of echocardiogram (echo) images according to 2D images and Doppler modality images;
classifying the 2D images by view type, including PLAX (parasternal long axis), apical 2-chamber (A2C), and apical 4-chamber (A4C);
classifying the Doppler modality images by region, including continuous wave of the mitral valve (CWMR) or continuous wave of the tricuspid valve (CWTR);
segmenting regions of interest in the 2D images to produce segmented 2D images, including PLAX segmented images, A2C segmented images, and A4C segmented images;
segmenting the Doppler modality images to generate waveform traces to produce segmented CW Doppler modality images, including CWMR or CWTR;
using both the segmented 2D images and the segmented CW Doppler modality images to calculate measurements of cardiac features of the heart;
generating a grade of MR or TR severity by comparing a portion of the calculated measurements to cardiac guidelines; and
outputting at least one report showing ones of the calculated measurements that fall within or outside of the cardiac guidelines.

2. The method of claim 1, providing the measurements of cardiac features of the heart comprise as MR parameters including: i) mitral valve vena contracta (VC) width comprising a mean of the measurements in the PLAX segmented images, the A2C segmented images, and the A4C segmented images; ii) mitral regurgitation jet area to left atrial area ratio (JAR); and iii) MR CW Doppler Density (CWDD).

3. The method of claim 2, providing the measurements of cardiac features of the heart comprise as TR parameters including: i) tricuspid valve vena contracta (VC) width in the A4C segmented images; ii) tricuspid regurgitation jet area (JA) in the A4C segmented images; and iii) TR CW Doppler Density (CWDD).

4. The method of claim 3 calculating the MR CWDD parameter and the TR CWDD parameter for a given CW image by generating a mask; assigning a numerical values in grayscale to pixels comprising the mask; normalizing the numerical values to a brightness of the entire CW waveform image to generate normalized brightness values; and calculating the MR CWDD or TR CWDD parameter as an average of the normalized brightness values.

5. The method of claim 3, wherein generating the grade of MR or TR severity further comprises classifying the MR or TR severity by determining a respective grade for each of the MR or TR parameters, wherein a highest grade for each of the MR or TR parameters is used to determine a final MR or TR severity grade.

6. The method of claim 1, wherein receiving, from a memory, the plurality of echo images, further comprises:
receiving the plurality of echo images directly from a local or remote source, including an ultrasound device;
storing the plurality of echo images in an image archive; and
opening the stored echo images in the memory for processing.

7. The method of claim 1, wherein separating the plurality of echo images further comprises:
analyzing metadata incorporated in the echo images to distinguish between the 2D and the Doppler modality images;
separating the Doppler modality images into either pulse wave, continuous wave, PWTDI or m-mode groupings;
performing color flow analysis on extracted pixel data using a combination of metadata and color content within the echo images to separate views that contain color from those that do not;
removing from the echo images any metatags that contain personal information and cropping the echo images to exclude any identifying information; and
extracting pixel data from the echo images and converting the pixel data to numpy arrays for further processing.

8. The method of claim 1, wherein classifying the 2D images and the Doppler modality images is based on a majority voting scheme comprising:
dividing a video of a 2D image of a Doppler modality image into frames;
generating for the frames, classification labels that constitute votes; and
applying a particular one of the classification labels receiving a highest number of the votes as the classification of the video.

9. The method of claim 1, further comprising: implementing the workflow engine to comprise a set of one or more classification convolutional neural networks (CNNs) for view classification, a set of one or more segmentation CNNs for chamber segmentation and waveform mask/trace, a set of one or more prediction CNNs for disease prediction.

10. The method of claim 9, wherein the one or more segmentation CNNs are trained from hand-labeled real images or artificial images generated by general adversarial networks (GANs).

11. The method of claim 1, further comprising: maintaining classification confidence scores, annotation confidence scores, and measurement confidence scores during processing and filtering out ones of the confidence scores failing to meet a threshold.

12. The method of claim 11, further comprising: for all non-filtered out data, selecting as best measurement data the measurements associated with cardiac chambers with largest volumes; and saving with the best measurement data, image location, classification, annotation and other measurement data associated with the best measurement data.

13. A system, comprising:
a memory storing a plurality of echocardiogram images of a heart;
at least one processor coupled to the memory;
a workflow engine, which when executed by the at least one processor is configurable to:
receive, from a memory, a plurality of echocardiogram images a patient heart;
separate the plurality of echocardiogram (echo) images according to 2D images and Doppler modality images;
classify the 2D images by view type, including PLAX (parasternal long axis), apical 2-chamber (A2C), and apical 4-chamber (A4C);
classify the Doppler modality images by region, including continuous wave of the mitral valve (CWMR) or continuous wave of the tricuspid valve (CWTR);
segment regions of interest in the 2D images to produce segmented 2D images, including PLAX segmented images, A2C segmented images, and A4C segmented images;
segment the Doppler modality images to generate waveform traces to produce segmented CW Doppler modality images, including CWMR or CWTR;
use both the segmented 2D images and the segmented CW Doppler modality images to calculate measurements of cardiac features of the heart;
generate a grade of MR or TR severity by comparing a portion of the calculated measurements to cardiac guidelines; and
output at least one report showing ones of the calculated measurements that fall within or outside of the cardiac guidelines.

14. The system, of claim 13, wherein the measurements of cardiac features of the heart comprise MR parameters including: i) mitral valve vena contracta (VC) width comprising a mean of the measurements in the PLAX segmented images, the A2C segmented images, and the A4C segmented images; ii) mitral regurgitation jet area to left atrial area ratio (JAR); and iii) MR CW Doppler Density (CWDD).

15. The system of claim 14, wherein the measurements of cardiac features of the heart comprise TR parameters including: i) tricuspid valve vena contracta (VC) width in the A4C segmented images; ii) tricuspid regurgitation jet area (JA) in the A4C segmented images; and iii) TR CW Doppler Density (CWDD).

16. The system of claim 15 wherein the MR CWDD parameter and the TR CWDD parameter are calculated for a given CW image by generating a mask; assigning a numerical values in grayscale to pixels comprising the mask; normalizing the numerical values to a brightness of the entire CW waveform image to generate normalized brightness values; and calculating the MR CWDD or TR CWDD as an average of the normalized brightness values.

17. The system of claim 15, wherein the grade of MR or TR severity are calculated by classifying the MR or TR severity by determining a respective grade for each of the MR or TR parameters, wherein a highest grade for each of the MR or TR parameters is used to determine a final MR or TR severity grade.

18. The system of claim 13, wherein the workflow engine receives from the plurality of echo images directly from a local or remote source, including an ultrasound device;
stores the plurality of echo images in an image archive; and
opens the stored echo images in the memory for processing.

19. The system of claim 13, wherein the workflow engine separates the plurality of echo images uses metadata incorporated in the echo images to distinguish between the 2D and the Doppler modality images;
separates the Doppler modality images into either pulse wave, continuous wave, PWTDI or m-mode groupings;
performs color flow analysis on extracted pixel data using a combination of metadata and color content within the echo images to separate views that contain color from those that do not;
removes from the echo images any metatags that contain personal information and cropping the echo images to exclude any identifying information; and
extracts pixel data from the echo images and converts the pixel data to numpy arrays for further processing.

20. The system of claim 13, wherein the workflow engine classifies the 2D images and the Doppler modality images based on a majority voting scheme, wherein workflow engine divides a video of a 2D image of a Doppler modality image into frames;
generates for the frames, classification labels that constitute votes; and
applies a particular one of the classification labels receiving a highest number of the votes as the classification of the video.

21. The system of claim 13, wherein the workflow engine is implemented to comprise a set of one or more classification convolutional neural networks (CNNs) for view classification, a set of one or more segmentation CNNs for chamber segmentation and waveform mask/trace, a set of one or more prediction CNNs for disease prediction.

22. The system of claim 21, wherein the one or more segmentation CNNs are trained from hand-labeled real images or artificial images generated by general adversarial networks (GANs).

23. The system of claim 13, wherein the workflow engine maintains classification confidence scores, annotation confidence scores, and measurement confidence scores during processing and filters out ones of the confidence scores failing to meet a threshold.

24. The system of claim 23, wherein for all non-filtered out data, the workflow engine selects as best measurement data the measurements associated with cardiac chambers with largest volumes; and saves with the best measurement data, image location, classification, annotation and other measurement data associated with the best measurement data.

25. An executable software product stored on a non-transitory computer-readable medium containing program instructions for implementing an automated workflow for grading of mitral valve regurgitation (MR) or tricuspid valve regurgitation (TR) severity, which when executed by a set of one or more processors, are configurable to cause the set of one or more processors to perform operations comprising:
receiving, from a memory, a plurality of echocardiogram images a heart;
separating the plurality of echocardiogram (echo) images according to 2D images and Doppler modality images;
classifying the 2D images by view type, including PLAX (parasternal long axis), apical 2-chamber (A2C), and apical 4-chamber (A4C);
classifying the Doppler modality images by region, including continuous wave of the mitral valve (CWMR) or continuous wave of the tricuspid valve (CWTR);
segmenting regions of interest in the 2D images to produce segmented 2D images, including PLAX segmented images, A2C segmented images, and A4C segmented images;
segmenting the Doppler modality images to generate waveform traces to produce segmented CW Doppler modality images, including CWMR or CWTR;
using both the segmented 2D images and the segmented CW Doppler modality images to calculate measurements of cardiac features of the heart;
generating a grade of MR or TR severity by comparing a portion of the calculated measurements to cardiac guidelines; and
outputting at least one report showing ones of the calculated measurements that fall within or outside of the cardiac guidelines.
